(19) United States
(12) Patent Application Publication
(10) Pub. No.: US 2023/0135724 A1
(43) Pub. Date: May 4, 2023

(54) ENDOSCOPE SYSTEM

(71) Applicant: FUJIFILM Corporation, Tokyo (JP)

(72) Inventor: Hiroaki TABE, Kanagawa (JP)

(73) Assignee: FUJIFILM Corporation, Tokyo (JP)

(21) Appl. No.: 18/148,912

(22) Filed: Dec. 30, 2022

Related U.S. Application Data
(63) Continuation of application No. PCT/JP21/11184, filed on Mar. 18, 2021.

(30) Foreign Application Priority Data
Jul. 8, 2020 (JP) 2020-117762

(51) Int. Cl. A61B 1/00; A61B 1/005; A61B 1/06

(52) U.S. Cl. CPC A61B 1/00096; A61B 1/00126; A61B 1/005; A61B 1/0638

(57) ABSTRACT

An endoscope includes an insertion part, a connector, and a light guide unit. A light source device includes a light source unit. The light guide unit includes light guides that guide illumination light emitted from the light source unit to a distal end portion of the insertion part in a case where the endoscope is connected to the light source device, and a lens member that suppresses a variation in a relative intensity of each color light of the illumination light, which is emitted from the distal end portion, with respect to a light distribution angle. The lens member is disposed in the connector having stiffness.

### CROSS-REFERENCE TO RELATED APPLICATIONS

This application is a Continuation of PCT International Application No. PCT/JP2021/011184 filed on 18 Mar. 2021, which claims priority under 35 U.S.C § 119(a) to Japanese Patent Application No. 2020-117762 filed on 8 Jul. 2020. The above application is hereby expressly incorporated by reference, in its entirety, into the present application.

### BACKGROUND OF THE INVENTION

### 1. Field of the Invention

The present invention relates to an endoscope system.

### 2. Description of the Related Art

Endoscopes are widely used in the medical field and the industrial field. An endoscope includes an insertion part to be inserted into an object to be examined, and irradiates an object to be observed with illumination light from a distal end portion of the insertion part. A light guide unit for guiding illumination light, which is supplied from a light source device, to the distal end portion of the insertion part is provided in the endoscope. The light guide unit guides illumination light, and the object to be observed is illuminated with the illumination light, so that the inside of the object to be examined can be observed.

In an endoscope disclosed in JP1994-296584A (JP-H06-296584A), a light guide unit comprises a plurality of light guides. The light guide is formed of, for example, a fiber bundle in which quartz fibers or multi-component fibers are bundled. Among these fiber bundles, a fiber bundle formed of quartz fibers is excellent in flexibility but has a high attenuation rate of light. Accordingly, in the endoscope disclosed in JP1994-296584A (JP-H06-296584A), a lens is disposed between the plurality of light guides. Since this lens is used, a reduction in the amount of light and deterioration in the distribution of light are prevented, and light emitted from an end portion of a light source-side light guide is transmitted to an end portion of a distal end portion-side light guide.

However, there is an endoscope in which a bendable part is provided at a distal end of an insertion part to allow a user to observe a portion to be observed at various angles or to easily insert the insertion part. In the endoscope disclosed in JP1994-296584A (JP-H06-296584A), the end portions of the light guides and the lens are disposed in a bendable part.

### SUMMARY OF THE INVENTION

However, since the end portions of the light guides and the lens are disposed in the bendable part in the endoscope disclosed in JP1994-296584A (JP-H06-296584A), there is a possibility that an optical axis is shifted. That is, in a case where the bendable part is bent, central axes of the end portions of the light source-side light guide and the distal end portion-side light guide facing the lens do not coincide with the central axis of the lens for preventing a reduction in the amount of light and deterioration in the distribution of light. For this reason, there is a high probability that deterioration in performance, such as a reduction in the amount of light and deterioration in the distribution of light, will occur.

An object of the present invention is to provide an endoscope system that can prevent a reduction in the amount of illumination light and deterioration in the distribution of the illumination light regardless of an operation state of an endoscope.

An endoscope system according to an aspect of the present invention comprises: an endoscope that includes an insertion part, an illumination light-emission end, and a light guide unit; and a light source device. The light guide unit includes a light guide and an optical member, and the optical member is provided in the endoscope at a portion that is closer to a proximal end side than to the illumination light-emission end and that has stiffness. The insertion part is to be inserted into an object to be examined. The illumination light-emission end is provided at a distal end portion of the insertion part. The light guide unit guides illumination light. The light source device is connected to the endoscope, includes a plurality of light sources emitting color lights different from each other, and emits illumination light in which a plurality of color lights are mixed by the light sources. The light guide includes a light guide that guides the illumination light emitted from the light sources to the illumination light-emission end in a case where the endoscope is connected to the light source device. The optical member suppresses a variation in a relative intensity of each color light of the illumination light, which is guided by the light guide and is emitted from the illumination light-emission end, with respect to a light distribution angle.

It is preferable that the light guide unit includes a plurality of the light guides and the optical member is a lens member disposed between the plurality of light guides.

It is preferable that the light guide and the lens member are disposed at a position where an emission point of the illumination light guided by the light guide is closer to the lens member than to a focal point of the lens member on an incident side.

It is preferable that lens surfaces of the lens member are disposed to have intervals from an emission end and an incident end of the light guides.

It is preferable that the endoscope includes a connector to be connected to the light source device, and the optical member is disposed in the connector. It is preferable that the connector includes a light guide rod that is to be inserted into the light source device in a case where the connector is connected to the light source device, and the optical member is disposed in the light guide rod.

It is preferable that the insertion part includes a bendable part that changes an orientation of the distal end portion, and the optical member is provided in the endoscope at a portion other than the bendable part.

It is preferable that the endoscope includes an operation part connected to the insertion part, and the optical member is provided in the operation part.

It is preferable that a dimension from an incident end to an emission end of a light guide, which is positioned on an incident side of the optical member, among the plurality of light guides in a direction of an optical axis is 5 mm or more. It is preferable that an antireflection film is formed on a lens surface of the lens member.

It is preferable that, in a case where a relative intensity of one color light among the plurality of color lights emitted from the light sources is used as a reference, the lens member makes a relative intensity of the other color light have a difference of ±5% or less from the relative intensity of the color light used as the reference.

According to the endoscope system of the aspect of the present invention, it is possible to prevent a reduction in the amount of illumination light and deterioration in the distribution of the illumination light regardless of an operation state of an endoscope.

### BRIEF DESCRIPTION OF THE DRAWINGS

FIG. 1 is an external view of an endoscope system.

FIG. 2 is a block diagram showing a schematic configuration of the endoscope system.

FIG. 3 is an external perspective view of a light source device.

FIG. 4 is an external perspective view of a connector.

FIG. 5 is a front view of the connector.

FIG. 6 is an exploded perspective view of the connector.

FIG. 7 is a cross-sectional view of a main portion of the connector.

FIG. 8 is a cross-sectional view of a main portion enlarging a part of FIG. 7 and showing a configuration around a lens member.

FIG. 9 is a perspective view of a spacer member, a lens member, and an O-ring.

FIG. 10 is a diagram illustrating a positional relationship between a first light guide, a second light guide, and a lens member.

FIG. 11 is an end view showing the shape of an end surface of an incident end of the first light guide.

FIG. 12 is an end view showing the shape of an end surface of an emission end of the second light guide.

FIG. 13 shows illumination characteristics showing the relative intensity of each color light of illumination light with respect to a light distribution angle at a position in a light guide unit where the illumination light has not yet passed through the lens member.

FIG. 14 shows illumination characteristics showing the relative intensity of each color light of illumination light with respect to a light distribution angle at a position in the light guide unit where the illumination light has passed through the lens member.

FIG. 15 is a cross-sectional view of a main portion showing a configuration of a connector of a second embodiment.

FIG. 16 is an external view showing a configuration of an endoscope of a third embodiment.

### DESCRIPTION OF THE PREFERRED EMBODIMENTS

### First Embodiment

[Schematic Configuration of Endoscope System]

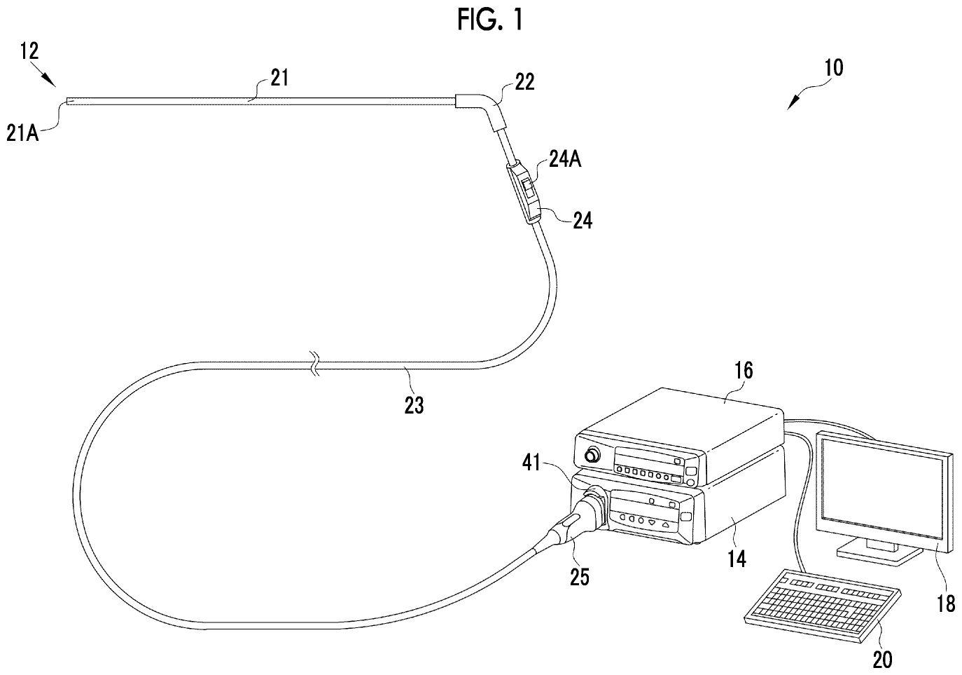

As shown in FIG. 1, an endoscope system 10 includes an endoscope 12, a light source device 14, a processor device 16, a monitor 18, and a console 20. The endoscope 12 is, for example, a rigid endoscope, such as a laparoscope, and comprises an elongated rigid insertion part 21 that is to be inserted into an object to be examined, an L-shaped grip part 22 that is connected to a proximal end portion of the insertion part 21, a soft universal cable 23 of which a distal end portion is connected to the insertion part 21 via the grip part 22, and a switch-disposition member 24 that is provided in a middle portion of the universal cable 23.

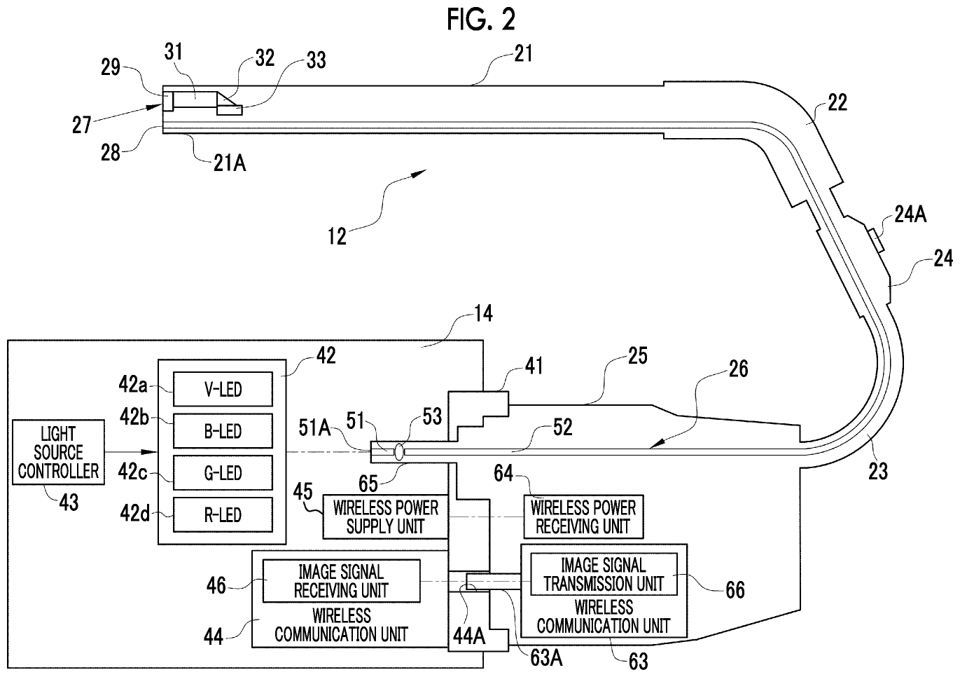

A connector 25 is provided at a proximal end portion of the universal cable 23, and the endoscope 12 is attachably and detachably connected to a light source device-side connector 41 of the light source device 14 via the connector 25. The universal cable 23 is a cable in which a light guide unit 26 (see FIG. 2) for guiding illumination light emitted from the light source device 14, a control line for controlling an image pickup sensor 33 provided in a distal end portion 21A of the insertion part 21, a signal line for transmitting image signals output from the image pickup sensor 33 in a case where an image of an object to be observed irradiated with illumination light is picked up, a power line for supplying power to each part, such as the image pickup sensor, and the like are integrated. The control line, the signal line, and the power line are not shown in FIG. 2 to reduce the complexity of FIG. 2.

The endoscope system 10 according to this embodiment has a configuration in which power, optical signals, and the like are transmitted between the endoscope 12 and the light source device 14 via the connector 25 and the light source device-side connector 41 in a contactless manner. Further, for example, an image selector switch, which is used to switch an image displayed on the monitor 18 to a normal picked-up image and a special light image (for example, a white light (WL) image, a blue laser imaging (BLI) image, a linked color imaging (LCI) image, or a low-oxygen imaging image), can be applied as an operation switch 24A disposed on the above-mentioned switch-disposition member 24. Furthermore, the operation switch is not limited thereto, and an image stop switch, an image pickup switch, a zoom switch comprising a telephoto button and a wide button, a washing switch for the distal end portion of the insertion part, a light amount-adjustment switch, a sensitivity-adjustment switch, or the like can also be applied.

[Schematic Configuration of Endoscope]

As shown in FIG. 2, the distal end portion 21A of the insertion part 21 is provided with an observation unit 27 and an illumination light-emission end (hereinafter, simply referred to as an emission end) 28. The observation unit 27 comprises an observation window 29, an image pickup lens group 31 and a prism 32 that are disposed behind the observation window 29, and the image pickup sensor 33.

The image pickup sensor 33 is, for example, a color sensor including primary color filters, and comprises three types of pixels, that is, B pixels (blue pixels) including blue color filters, G pixels (green pixels) including green color filters, and R pixels (red pixels) including red color filters. The blue color filter mainly transmits violet to blue light. The green color filter mainly transmits green light. The red color filter mainly transmits red light. In a case where the image of the object to be observed is picked up using the primary color image pickup sensor 33 as described above, a maximum of three types of images, that is, a B image (blue image) obtained from B pixels, a G image (green image) obtained from G pixels, and an R image (red image) obtained from R pixels, can be simultaneously obtained.

A charge coupled device (CCD) image sensor or a complementary metal oxide semiconductor (CMOS) image sensor can be applied as the image pickup sensor 33. Further, the image pickup sensor 33 of this embodiment is a primary color sensor, but a complementary color sensor can also be used. A complementary color sensor includes, for example, cyan pixels provided with cyan color filters, magenta pixels provided with magenta color filters, yellow pixels provided with yellow color filters, and green pixels provided with green color filters. In a case where a complementary color sensor is used, images obtained from the respective color pixels described above can be converted into B images, G images, and R images by complementary color-primary color conversion. Further, a monochrome sensor not provided with color filters may be used as the image pickup sensor 33 instead of the color sensor. In this case, the images having the respective colors can be obtained from the sequential image pickup of the object to be observed using illumination light having the respective colors, such as B, G, and R.

[Schematic Configuration of Processor Device]

The processor device 16 controls the amount and emission time of illumination light that is emitted from the light source device 14, the operation of the image pickup sensor 33, and the like, and generates an endoscopic image using image signals that are obtained from the image pickup of the object to be observed irradiated with illumination light. The processor device 16 is electrically connected to the monitor 18 and to the console 20. The monitor 18 displays the endoscopic image generated by the processor device 16, information about the endoscopic image, and the like. The console 20 is a user interface that receives an input operation, such as function settings.

[Schematic Configuration of Light Source Device]

As shown in FIG. 2, the light source device 14 comprises a light source unit 42, a light source controller 43, a wireless communication unit 44, and a wireless power supply unit 45. The light source unit 42 emits illumination light that is used to illuminate the object to be observed. The light source controller 43 controls the light source unit 42. Although not shown, the light source device 14 comprises the processor device 16 and a signal transmission unit that transmits control signals, image signals, and the like.

The light source unit 42 includes semiconductor light sources, such as a plurality of color light emitting diodes (LEDs). The light source controller 43 adjusts the turning on or off of the LEDs or the drive currents or drive voltages of the LEDs to control the amount of illumination light to be emitted. The semiconductor light sources of the light source unit 42 are not limited to LEDs and may be laser diodes (LDs) or the like.

The light source unit 42 includes four color LEDs, that is, a violet light emitting diode (V-LED) 42a, a blue light emitting diode (B-LED) 42b, a green light emitting diode (G-LED) 42c, and a red light emitting diode (R-LED) 42d.

The LEDs 42a to 42d emit color lights different from each other. For example, the V-LED 42a emits a violet light V having a wavelength range of 380 nm to 420 nm. The B-LED 42b emits a blue light B having a wavelength range of 420 nm to 500 nm. The G-LED 42c emits a green light G having a wavelength range of 480 nm to 600 nm. The R-LED 42d emits a red light R having a wavelength range of 600 nm to 650 nm. Lights emitted from the LEDs 42a to 42d may have the same central wavelength and the same peak wavelength, and may have different central wavelengths and different peak wavelengths.

The light source controller 43 independently controls the turning on or off of the respective LEDs 42a to 42d, the amounts of lights emitted at the times of turning on thereof, and the like to adjust the emission time, emission period, amount, and spectrum of illumination light. The control of the turning on or off of each of the LEDs, which is performed by the light source controller 43, varies depending on each observation mode. Reference brightness can be set by the console 20 or the like.

In the case of a normal mode, the light source controller 43 turns on all the V-LED 42a, the B-LED 42b, the G-LED 42c, and the R-LED 42d. Accordingly, in the normal mode, polychromatic light for a normal mode, which includes a violet light, a blue light, a green light, and a red light, is emitted from the light source device 14 as normal light. Since a violet light, a blue light, a green light, and a red light are mixed in the normal light and the normal light has an intensity equal to or higher than a certain intensity from a blue light wavelength range to a red light wavelength range, the normal light is a substantially white light. A white light includes not only broadband light that includes all the wavelength ranges of a blue light component, a green light component, and a red light component like a white light emitted from a xenon lamp, but also illumination light in which lights having wavelength ranges of at least three color light components, such as a blue light component, a green light component, and a red light component, are mixed.

In the case of a special mode, the light source controller 43 turns on all the V-LED 42a, the B-LED 42b, the G-LED 42c, and the R-LED 42d but sets a light amount ratio between a violet light, a blue light, a green light, and a red light in this case such that a ratio of a violet light is increased. Accordingly, a special light is a bluish light. The case of the normal mode in which a white light is emitted as illumination light will be mainly described below.

Further, the light source device 14 is electrically connected to the processor device 16, and the connector 25 of the endoscope 12 is connected to the processor device 16 via the light source device 14. The transmission and reception of image signals and the like between the light source device 14 and the connector 25 are via wireless communication. For this reason, the light source device 14 transmits image signals and the like, which are transmitted to and received from the connector 25 wirelessly, to the processor device 16. Furthermore, the light source device 14 supplies power, which is used to drive the image pickup sensor 33 and the like, to the connector 25 but also supplies this power wirelessly.

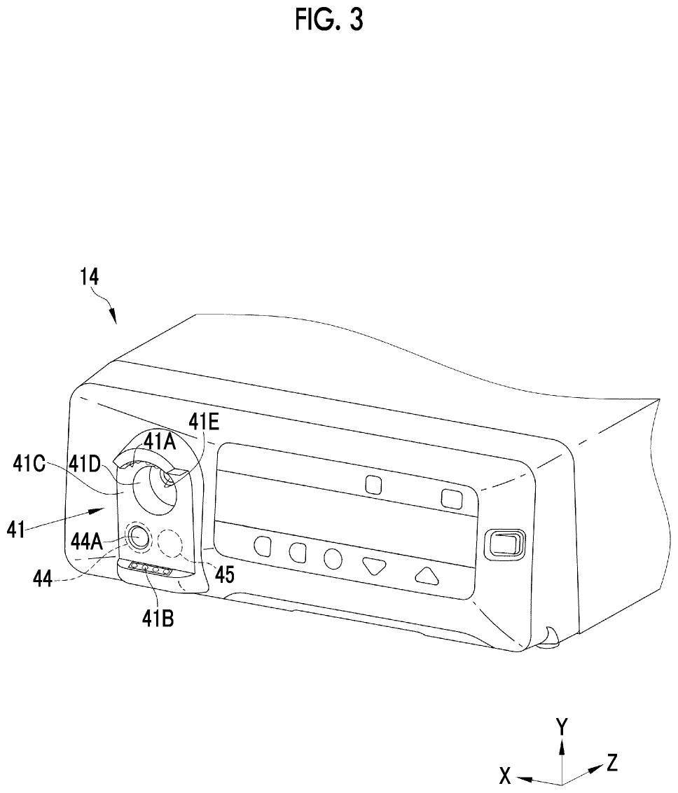

As shown in FIG. 3, the light source device-side connector 41 is provided with locking portions 41A and 41B, a contact surface 41C, a fitting recess 41D, and a connection hole 41E. The contact surface 41C is a flat surface that is in contact with a front surface 61A of the connector 25 to be described later. The locking portions 41A and 41B are provided at positions protruding from the contact surface 41C. In a case where the connector 25 is connected, the locking portions 41A and 41B are locked to the connector 25 and maintain connection. The fitting recess 41D is a recess that is recessed from the contact surface 41C by one step, and a fitting projection 61B of the connector 25 to be described later is fitted to the fitting recess 41D. The connection hole 41E is a through-hole provided in the fitting recess 41D, and a light guide rod 65 of the connector 25 is inserted into the connection hole 41E.

In a case where the connector 25 is connected to the light source device-side connector 41, the light guide rod 65 is inserted into the connection hole 41E of the light source device-side connector 41, so that an incident end 51A of a first light guide 51 faces the light source unit 42 of the light source device 14. Accordingly, illumination light emitted from the light source unit 42 is transmitted via the first light guide 51 and a lens member 53, and is emitted to a front side of the insertion part 21 from an emission end 28 of a second light guide 52.

Further, the wireless communication unit 44 that wirelessly communicates with a wireless communication unit 63 of the connector 25 and the wireless power supply unit 45 that supplies power to a wireless power receiving unit 64 of the connector 25 are provided behind a lower portion of the contact surface 41C. The wireless communication unit 44 includes an image signal receiving unit 46 (see FIG. 2). The image signal receiving unit 46 receives image signals from an image signal transmission unit 66 of the connector 25. A connection hole 44A of the wireless communication unit 44 is formed of a hole portion having a shape that allows a connection pin 63A of the connector 25 to be inserted. The wireless power supply unit 45 is, for example, a coil (so-called primary coil), and supplies power to the wireless power receiving unit 64 with a contactless power transmission method, such as an electromagnetic induction method or a magnetic resonance method.

In a case where the connector 25 of the endoscope 12 is connected to the light source device-side connector 41 of the light source device 14, illumination light emitted from the light source unit 42 is incident on the light guide unit 26 of the endoscope 12 via, for example, light guide members (not shown), such as a prism and a light guide rod.

The light guide unit 26 is built in the endoscope 12 including the universal cable 23 and the connector 25, and guides illumination light up to the distal end portion 21A of the endoscope 12. The distal end portion 21A is provided with the emission end 28. The emission end 28 is disposed around the observation window 29, and is a distal end of the second light guide 52 to be described later. In this embodiment, the observation window 29 and the emission end 28 are exposed from a distal end surface of the distal end portion 21A. Illumination light emitted from the light source unit 42 is guided from the light source unit 42 by the light guide unit 26 and is applied to the object to be observed from the emission end 28.

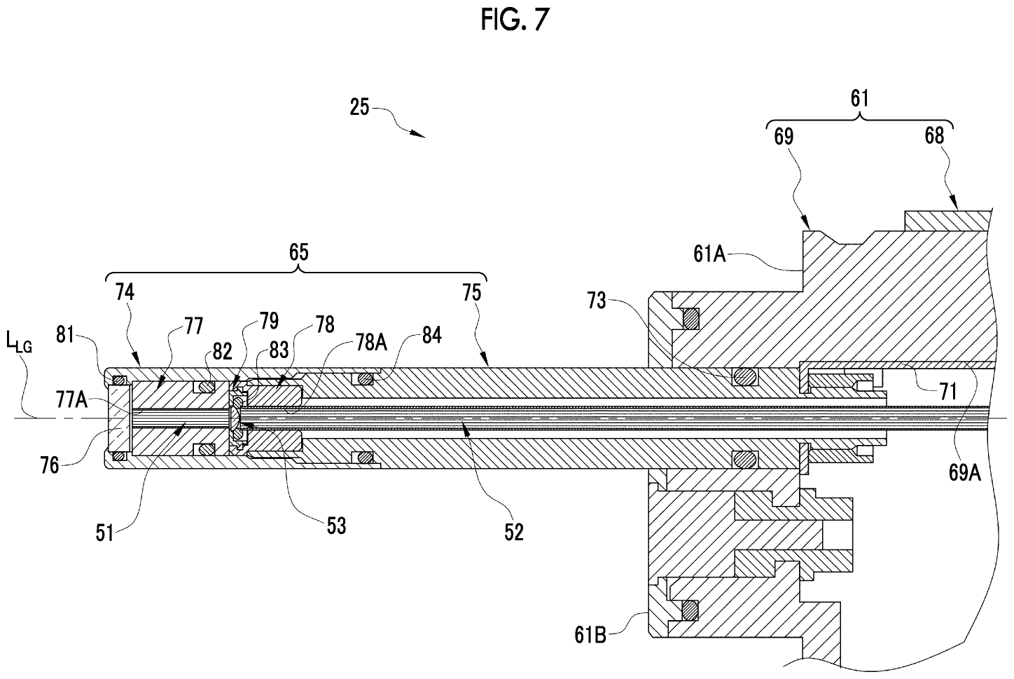

The light guide unit 26 includes the first light guide 51, the second light guide 52, and the lens member 53. Each of the light guides 51 and 52 is a fiber bundle in which optical fibers are bundled. The lens member 53 corresponds to an optical member of the claims. The optical fibers forming the light guides 51 and 52 are, for example, quartz fibers or multi-component fibers. In this embodiment, the first light guide 51, a part of the second light guide 52, and the lens member 53 are provided in the connector 25 and are more specifically provided in the light guide rod 65 (see FIG. 7). The structure of the light guide rod 65 will be described later.

[Schematic Configuration of Connector]

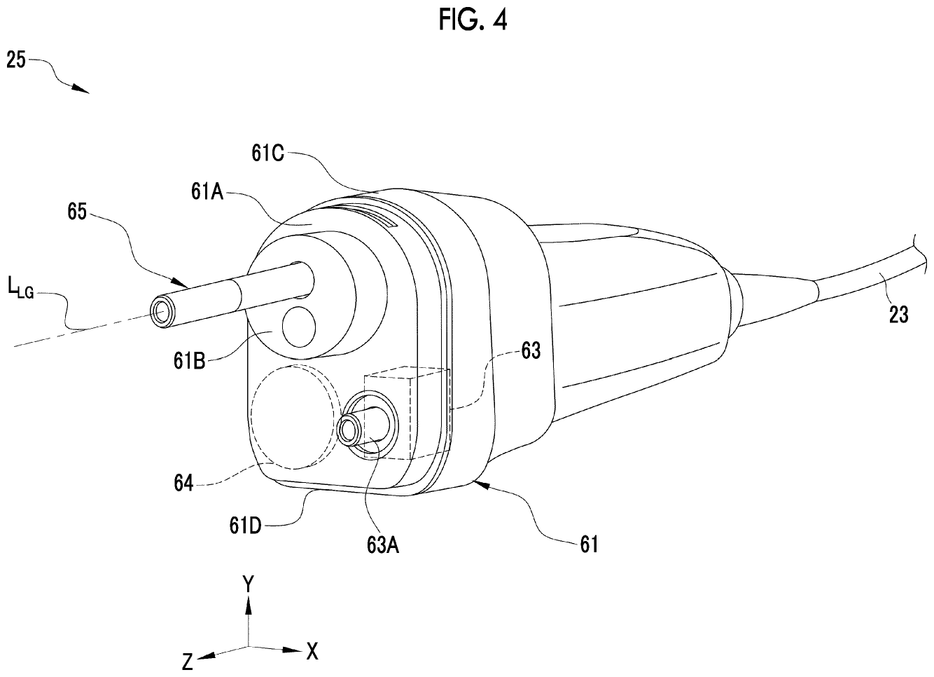

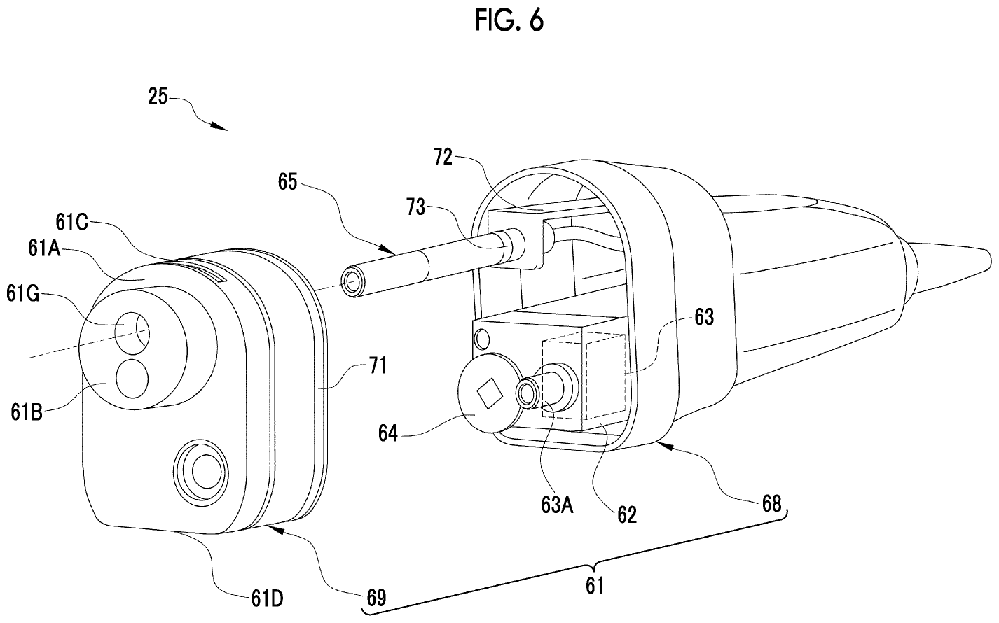

As shown in FIG. 4, the connector 25 includes an outer case 61, a shield case 62 (see FIG. 6), the wireless communication unit 63, the wireless power receiving unit 64, and the light guide rod 65. Hereinafter, a posture in which the connector 25 is correctly connected to the light source device 14 is defined as “connection posture”, a connection direction in which the connector 25 is connected to the light source device 14 in the connection posture is defined as a Z direction, a vertical direction of the connector 25 in the connection posture is defined as a Y direction, and a direction in a horizontal plane perpendicular to the Z direction and to the Y direction is defined as an X direction. A positive side in the Z direction is defined as a side on which the light source device 14 is positioned as viewed from the connector 25, a positive side in the Y direction is defined as a vertical upper side, and a positive side in the X direction is defined as a left side as viewed from a negative side in the Z direction. Hereinafter, the positive side in the Z direction, which is the connection direction of the connector 25, may be referred to as a proximal end side or a proximal end portion, and a negative side in the Z direction may be referred to as a distal end side or a distal end portion.

The outer case 61 houses the wireless communication unit 63 and the wireless power receiving unit 64, and the light guide rod 65 protrudes from the front surface 61A that is positioned on a side facing the light source device 14 in the connection posture. The wireless communication unit 63 wirelessly communicates with the light source device 14. The wireless power receiving unit 64 receives power that is wirelessly supplied from the light source device 14. Since a position where the light guide rod 65 protrudes is present on a vertical upper side in the connection posture, the outer case 61 houses the wireless communication unit 63 and the wireless power receiving unit 64 on a vertical lower side in the connection posture.

The fitting projection 61B, which is to be fitted to the fitting recess 41D of the light source device-side connector 41, is provided at a proximal end portion of the light guide rod 65, and the fitting projection 61B protrudes from the front surface 61A. A central axis L_{LG} of the light guide rod 65 is parallel to the Z direction.

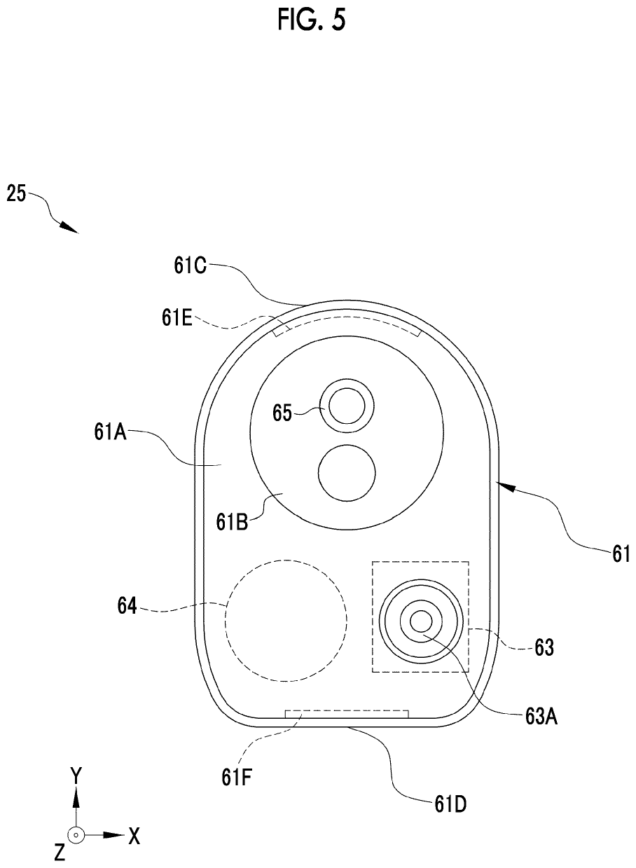

As shown in FIG. 5, the light guide rod 65 is disposed in the middle of the front surface 61A in the X direction. Further, an upper surface 61C of the outer case 61, which is positioned on the vertical upper side in the connection posture, is a curved surface, and a lower surface 61D thereof, which is positioned on a vertical lower side in the connection posture, is a flat surface. The light guide rod 65 is provided on a side of the front surface 61A close to the upper surface 61C. In contrast, the wireless communication unit 63 and the wireless power receiving unit 64 are housed to be arranged on the right and left on a side of the front surface 61A close to the lower surface 61D.

The wireless communication unit 63 includes the image signal transmission unit 66 (see FIG. 2). Electronic components, such as a substrate, of the image signal transmission unit 66 are housed in the shield case 62 made of metal. The image signal transmission unit 66 wirelessly transmits image signals, which are obtained from the image pickup of the object to be observed irradiated with illumination light, to the image signal receiving unit 46 of the light source device 14. Wireless communication performed by the wireless communication unit 63 is optical communication, and it is preferable that this wireless communication is, for example, near-infrared communication using near-infrared light (light having a wavelength of about 0.7 μm to 2.5 μm).

The wireless communication unit 63 includes the connection pin 63A. The connection pin 63A includes an emission end for optical signals at a distal end portion thereof, and can transmit and receive optical signals to and from the wireless communication unit 44 of the light source device 14 in a case where the connection pin 63A is connected to the connection hole 44A of the light source device-side connector 41. Accordingly, image signals of the image signal transmission unit 66 are optically transmitted to the image signal receiving unit 46 of the light source device 14 in a contactless manner. The image signals optically transmitted to the image signal receiving unit 46 are transmitted to the processor device 16. The image signals, which are transmitted to the processor device 16 from the endoscope 12 via the light source device 14, are subjected to image processing and are displayed on the monitor 18 as an endoscopic image. The functions of the wireless communication unit 44 of the light source device 14 and the wireless communication unit 63 of the connector 25 are not limited to the above-mentioned functions, and may be to transmit and receive control signals that are used to control, for example, the image pickup sensor 33 and the like of the endoscope 12.

The wireless power receiving unit 64 is, for example, a coil (so-called secondary coil), and receives power that is supplied from the wireless power supply unit 45 provided in the light source device 14 with a contactless power transmission method.

The wireless power receiving unit 64 is fixed to the shield case 62. Since an endoscope which supplies power using a primary coil and a secondary coil is publicly known in JP2016-67534A, the detailed description thereof will be omitted here. Since the wireless power receiving unit 64 is provided behind the front surface 61A as shown in FIG. 5, the wireless power receiving unit 64 receives power supplied from the light source device 14 through the front surface 61A. The wireless power receiving unit 64 supplies power to each part of the endoscope 12, such as the image pickup sensor 33.

In a case where the connector 25 is connected to the light source device-side connector 41, the connector 25 is locked at the upper surface 61C and at the lower surface 61D of the outer case 61, and connection is maintained. As described above, the light source device-side connector 41 is provided with the locking portions 41A and 41B. Correspondingly, a groove 61E to which the locking portion 41A of the light source device 14 is to be fitted is provided on the upper surface 61C of the connector 25, and a groove 61F to which the locking portion 41B of the light source device 14 is to be fitted is provided on the lower surface 61D of the connector 25.

As shown in FIG. 6, the outer case 61 includes a case body 68, and a case lid 69 and the case lid 69 is combined with a proximal end portion of the case body 68, so that the outer case 61 is formed in a hollow shape. The case body 68 and the case lid 69 of the outer case 61 are made of a resin having high heat resistance and high chemical resistance.

The case lid 69 is provided with the front surface 61A and the fitting projection 61B described above, and includes a part of the upper surface 61C and the lower surface 61D. An O-ring 71, which is a sealing member, is fitted to an outer peripheral surface of a rear end portion of the case lid 69. The O-ring 71 is one of a plurality of sealing members that seal the inside of the outer case 61.

A lead-out hole 61G, through which the light guide rod 65 is led to the outside, is formed at a central portion of the fitting projection 61B. The proximal end portion of the light guide rod 65 is fixed to a bracket 72 that is disposed in the outer case 61. The bracket 72 is formed in an L shape, and is fixed to the inside of the case body 68. The bracket 72 is fixed to an inner peripheral surface 69A (see FIG. 7) of the case lid 69, so that the light guide rod 65 is supported by the case body 68 and the case lid 69.

An elastic O-ring 73 is fitted to an outer peripheral surface of the light guide rod 65, and the light guide rod 65 is fitted to the lead-out hole 61G via the O-ring 73. As with the O-ring 71, the O-ring 73 is also one of a plurality of sealing members that seal the inside of the outer case 61.

[Schematic Configuration of Light Guide Rod]

As shown in FIG. 7, the light guide rod 65 comprises a first light guide rod 74 and a second light guide rod 75. The first light guide rod 74 and the second light guide rod 75 are made of metal or the like having high stiffness and have a cylindrical shape. The first light guide rod 74 houses an incident window 76, the first light guide 51, the second light guide 52, and the lens member 53 therein. The incident window 76 is a transparent cover glass and is fixed to a proximal end portion of the first light guide rod 74. Illumination light emitted from the light source unit 42 passes through the incident window 76, and is incident on the first light guide 51. An elastic O-ring 81 is fitted to an inner peripheral surface of the proximal end portion of the first light guide rod 74. The O-ring 81 is deformed between the incident window 76 and the first light guide rod 74 and is in close contact with both the incident window 76 and the first light guide rod 74, so that airtightness at the proximal end portion of the first light guide rod 74 is ensured.

The first light guide 51, the second light guide 52, and the lens member 53 are fixed in the first light guide rod 74 in a state where the first light guide 51 is held by a first ferrule 77, the second light guide 52 is held by a second ferrule 78, and the lens member 53 is held by a spacer 79.

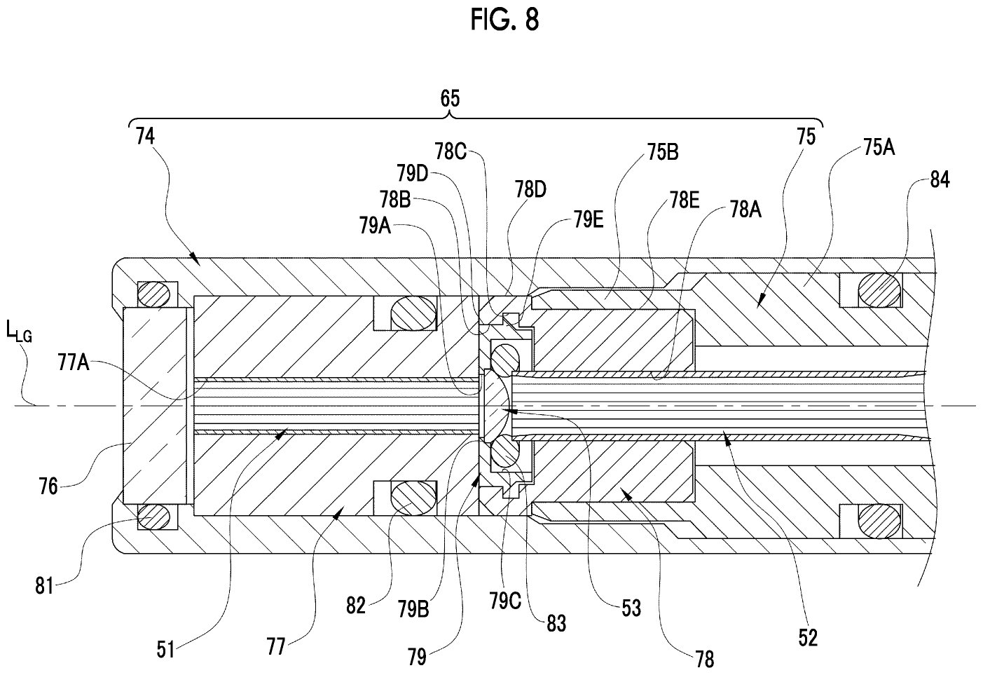

As shown in FIG. 8, the first ferrule 77 is formed in the shape of a cylinder in which a light guide-insertion hole 77A penetrating in an axial direction is provided at the center thereof. The first light guide 51 is inserted into the light guide-insertion hole 77A. The first light guide 51 is fixed to the light guide-insertion hole 77A by being adhered with, for example, an adhesive or the like. An elastic O-ring 82 is fitted to an outer peripheral surface of the first ferrule 77.

The first ferrule 77 is inserted up to a position close to the incident window 76 from the distal end side of the first light guide rod 74 together with the first light guide 51 and the O-ring 82, and is fitted to the inner peripheral surface of the first light guide rod 74. Accordingly, the first light guide 51 is fixed to the first light guide rod 74 together with the first ferrule 77. Further, the O-ring 82 is deformed between the first ferrule 77 and the first light guide rod 74 and is in close contact with both the first ferrule 77 and the first light guide rod 74, so that airtightness between the first ferrule 77 and the first light guide rod 74 is ensured.

The second ferrule 78 is formed in the shape of a cylinder in which a light guide-insertion hole 78A penetrating in an axial direction is provided at the center thereof. A proximal end portion of the second light guide 52 is inserted into the light guide-insertion hole 78A. The second light guide 52 protrudes from the distal end side of the light guide-insertion hole 78A, and is fixed to the distal end portion 21A of the endoscope 12 through the second light guide rod 75, the connector 25, the universal cable 23, the grip part 22, and the insertion part 21.

The proximal end portion of the second light guide 52 is fixed to the light guide-insertion hole 78A by being adhered with, for example, an adhesive or the like. An opening portion 78B is provided on a proximal end surface of the second ferrule 78 facing the first ferrule 77. The opening portion 78B is a circular opening portion that is formed to have an inner diameter larger than the inner diameter of the light guide-insertion hole 78A. The spacer 79 is fitted to the opening portion 78B. Further, a locking groove 78C is formed in the opening portion 78B.

A large-diameter portion 78D that has a large outer diameter and a small-diameter portion 78E that has an outer diameter smaller than the outer diameter of the large-diameter portion 78D are formed on an outer peripheral surface of the second ferrule 78. The large-diameter portion 78D is fitted to the inner peripheral surface of the first light guide rod 74. Accordingly, the proximal end portion of the second light guide 52 is fixed to the first light guide rod 74 together with the second ferrule 78. A proximal end of the second light guide 52 protrudes into the opening portion 78B from the proximal end side of the light guide-insertion hole 78A. Further, the small-diameter portion 78E has a gap between the small diameter portion 78E and the inner peripheral surface of the first light guide rod 74.

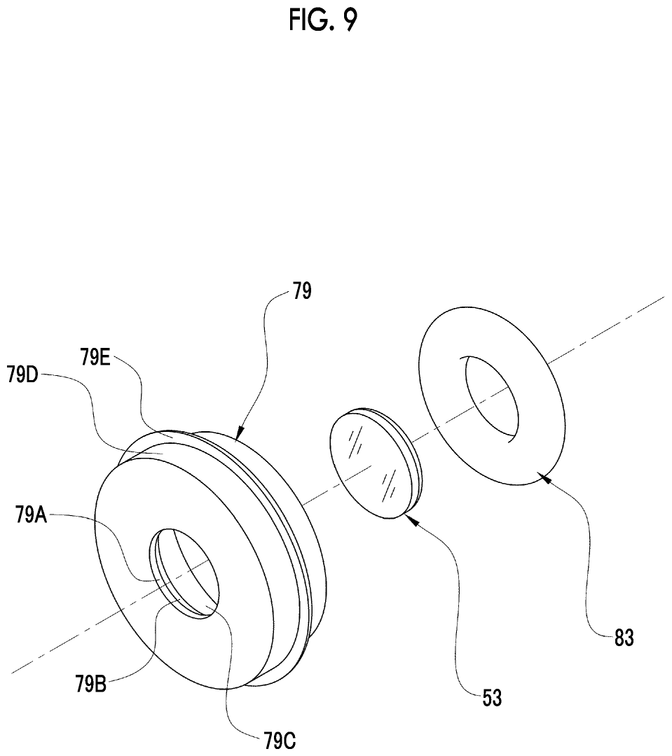

As shown in FIG. 9, the spacer 79 is formed in the shape of a disc in which a through-hole 79A penetrating in an axial direction is provided at the center thereof. The spacer 79 includes a lens member holding portion 79B and an O-ring housing portion 79C on the distal end side of the through-hole 79A. The lens member holding portion 79B is an opening portion that is slightly larger than the through-hole 79A, and is formed to have an inner diameter corresponding to the outer diameter of the lens member 53. An outer peripheral surface of the lens member 53 is fitted to the lens member holding portion 79B, so that the lens member holding portion 79B holds the lens member 53.

The O-ring housing portion 79C is an opening portion that is formed to have an inner diameter slightly larger than the inner diameter of the lens member holding portion 79B. The O-ring housing portion 79C houses an elastic O-ring 83 therein. The inner diameter of the O-ring 83 is smaller than the outer diameter of the lens member 53 and the outer diameter of the proximal end portion of the second light guide 52.

A locked portion 79E is formed on an outer peripheral surface 79D of the spacer 79. The locked portion 79E may be an annular protrusion along the outer peripheral surface 79D, or may be a plurality of protrusions protruding from the outer peripheral surface 79D. The locked portion 79E is locked to the locking groove 78C of the opening portion 78B. The outer peripheral surface 79D of the spacer 79 is formed to correspond to an inner peripheral surface of the opening portion 78B.

The spacer 79 is fixed to the opening portion 78B of the second ferrule 78 in a state where the lens member 53 is held in the lens member holding portion 79B and the O-ring 83 is housed in the O-ring housing portion 79C. The outer peripheral surface 79D is fitted to the inner peripheral surface of the opening portion 78B, and the locked portion 79E is locked to the locking groove 78C. Accordingly, since the position of the spacer 79 relative to the opening portion 78B is restricted, the spacer 79 is not separated from the opening portion 78B.

As described above, the proximal end of the second light guide 52 protrudes into the opening portion 78B. For this reason, in a case where the spacer 79 is fixed to the opening portion 78B of the second ferrule 78, the proximal end of the second light guide 52 protrudes into the spacer 79 and presses the O-ring 83 to the proximal end side. Since the lens member 53 is pressed by the second light guide 52 via the O-ring 83, the lens member 53 is pushed to the proximal end side of the spacer 79.

The lens member 53 is fixed to the second ferrule 78 via the spacer 79, and the second ferrule 78 is fitted to the first light guide rod 74, so that the lens member 53 is fixed to the first light guide rod 74. The lens member 53 is pressed toward the proximal end side by the O-ring 83 and is in contact with the edge of the through-hole 79A. Accordingly, the position of the lens member 53 relative to the spacer 79 in the axial direction is restricted.

As shown in FIG. 8, the second light guide rod 75 is combined with the distal end side of the first light guide rod 74. The second light guide rod 75 includes a fitting portion 75A at a proximal end portion thereof. The fitting portion 75A is formed to have an outer diameter smaller than the outer diameter of an outer peripheral surface of a portion of the second light guide rod 75 having the largest outer diameter, and is fitted to the inner peripheral surface of the first light guide rod 74. Further, an end portion 75B of the fitting portion 75A closest to the proximal end side is formed to have a small thickness, and is inserted into the gap between the small-diameter portion 78E of the second ferrule 78 and the inner peripheral surface of the first light guide rod 74.

The fitting portion 75A and the inner peripheral surface of the first light guide rod 74 are fitted to each other, so that the first light guide rod 74 and the second light guide rod 75 are combined with each other and the position of the second ferrule 78 is restricted. Accordingly, the second ferrule 78 is not separated to the distal end side. Combining the first light guide rod 74 with the second light guide rod 75 is not limited thereto, and an adhesive, a connection member, or the like may be used to connect both the first light guide rod 74 and the second light guide rod 75.

Further, an elastic O-ring 84 is fitted to the fitting portion 75A. The O-ring 84 is deformed between the inner peripheral surface of the first light guide rod 74 and the fitting portion 75A and is in close contact with both the inner peripheral surface of the first light guide rod 74 and the fitting portion 75A, so that airtightness between the first light guide rod 74 and the second light guide rod 75 is ensured. Since the O-rings 81, 82, and 84, and the like are mounted on the first light guide rod 74 and on the second light guide rod 75 as described above, saturated water vapor having high pressure and high temperature is prevented from entering the first light guide rod 74 and the second light guide rod 75 in a case where the endoscope 12 is subjected to sterilization treatment using an autoclave.

As described above, the first light guide 51, the second light guide 52, and the lens member 53 are fixed in the first light guide rod 74 via the first ferrule 77, the second ferrule 78, and the spacer 79. Accordingly, the lens member 53 is disposed between the first and second light guides 51 and 52. Further, since the first light guide 51, the second light guide 52, and the lens member 53 are held at the centers of the first ferrule 77, the second ferrule 78, and the spacer 79, respectively, the central axis L_{LG} of the light guide rod 65 coincides with the central axes of the first light guide 51, the second light guide 52, and the lens member 53.

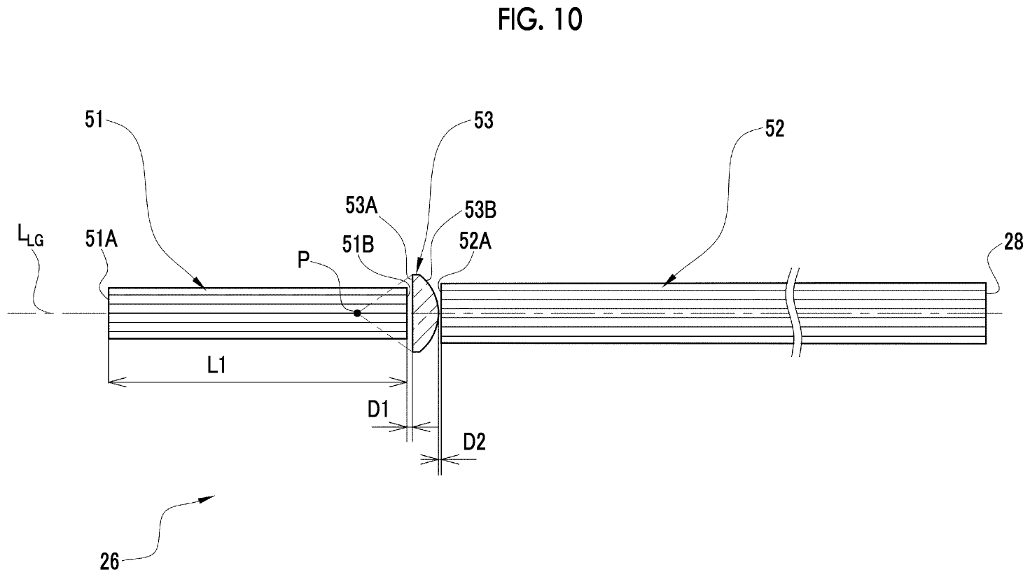

As shown in FIG. 10, the lens member 53 is a plano-convex lens of which an incident side-lens surface 53A is planar and an emission side-lens surface 53B is convex. The lens member 53 may be a biconvex lens, a meniscus lens, or the like without being limited thereto. It is preferable that an antireflection film called an anti-reflective (AR) coating is formed on each of the lens surfaces 53A and 53B of the lens member 53. Accordingly, a reduction in the amount of illumination light in the light guide unit 26 can be further prevented. FIG. 10 is a diagram illustrating a positional relationship between the first light guide 51, the second light guide 52, and the lens member 53 of the light guide unit 26, and the coatings and the like of the first light guide 51 and the second light guide 52 are not shown in FIG. 10 in order to reduce the complexity of the drawing.

Since the lens surface 53A of the lens member 53 is in contact with the edge of the through-hole 79A and the lens surface 53B of the lens member 53 is pressed from the O-ring 83, the position of the lens member 53 in the axial direction is restricted. Accordingly, the lens surfaces 53A and 53B of the lens member 53 are disposed to have intervals from an emission end 51B of the first light guide 51 and an incident end 52A of the second light guide 52. Specifically, the lens surface 53A has a gap D1 from the emission end 51B of the first light guide 51. It is preferable that the gap D1 is larger than 0 mm and 0.5 mm or less.

Meanwhile, since at least a part of the O-ring 83 is interposed between the lens member 53 and the second light guide 52, the lens surface 53B has a gap D2 from the incident end 52A of the second light guide 52. It is preferable that the gap D2 is larger than 0 mm and 0.5 mm or less.

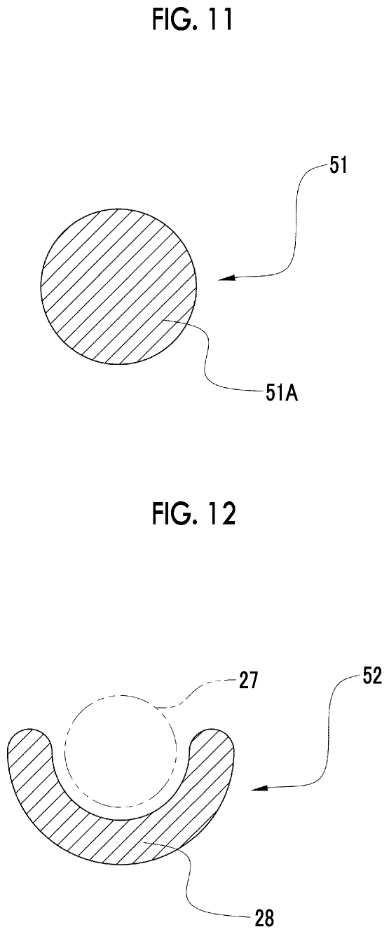

In the light guide unit 26 of this embodiment, the incident end 51A of the first light guide 51 facing the light source unit 42 is a circular end surface as shown in FIG. 11, but the emission end 28 of the second light guide 52 at the distal end portion 21A is formed in an arc shape positioned around the observation unit 27 as shown in FIG. 12 for the convenience of the disposition of components at the distal end portion 21A. Accordingly, it is difficult to dispose the lens member 53 at the distal end portion 21A. On the other hand, since there is no component that hinders the disposition of the lens member 53 in the light guide rod 65, it is easy to dispose the lens member 53 in the light guide rod 65.

The first light guide 51 and the lens member 53 are disposed at a position where an emission point of the first light guide 51 is closer to the lens surface 53A than to a focal point P of the lens member 53 on an incident side. Accordingly, after illumination light emitted from the emission point of the first light guide 51 passes through the lens member 53, the illumination light travels in a direction where the illumination light is diffused. Accordingly, the light guide unit 26 using the lens member 53 can obtain effects of preventing a reduction in the amount of light and preventing deterioration in the distribution of light. The first light guide 51 forms an emission point at the emission end 51B. For this reason, it is preferable that a dimension L1 from the incident end 51A to the emission end 51B of the first light guide 51 in the axial direction is 5 mm or more.

The first and second light guides 51 and 52 guide illumination light emitted from the light source unit 42 to the emission end 28 in a case where the connector 25 is connected to the light source device-side connector 41 of the light source device 14 as described above. As described above, the lens member 53 is a lens for preventing a reduction in the amount of illumination light, which is guided by the first and second light guides 51 and 52 and is emitted from the emission end 28, and deterioration in the distribution of the illumination light. Specifically, the lens member 53 is a lens for suppressing a variation in the relative intensity of each color light of the illumination light, which is guided by the first and second light guides 51 and 52 and is emitted from the emission end 28, with respect to a light distribution angle.

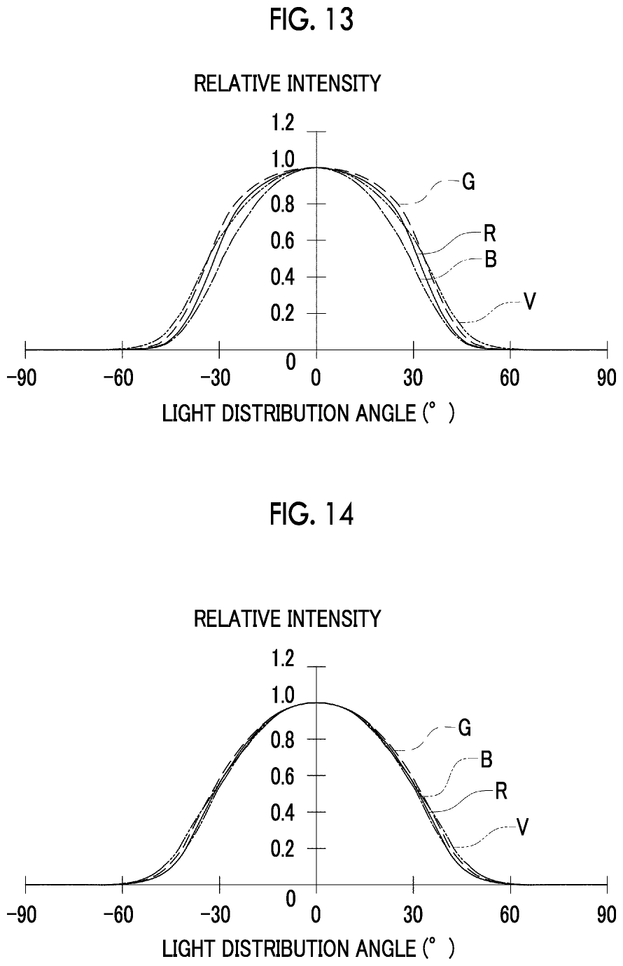

Graphs shown in FIGS. 13 and 14 are measurement results of illumination characteristics showing the relative intensity of each color light (LED light) of the illumination light with respect to a light distribution angle at a position (FIG. 13) in the light guide unit 26 where the illumination light has not yet passed through the lens member 53 and at a position (FIG. 14) in the light guide unit 26 where the illumination light has passed through the lens member 53. The relative intensity mentioned here is a ratio of the intensity of each color LED light at a light distribution angle other than 0° to the intensity of each color LED light at a light distribution angle of 0° in a case where the intensity of each color LED light at a light distribution angle of 0° is set as 1. The intensity of light is the density of the luminous flux of light within a unit solid angle.

As shown in FIG. 13, a violet light V, a blue light B, a green light G, and a red light R, which are LED lights emitted from the light source unit 42 and guided by the light guide unit 26, have variations in relative intensity with respect to a light distribution angle at a position where the illumination light has not yet passed through the lens member 53, specifically, at the emission end 51B of the first light guide 51. Particularly, a variation in relative intensity at a light distribution angle of about ±25° is large.

On the other hand, as shown in FIG. 14, a violet light V, a blue light B, a green light G, and a red light R, which are LED lights emitted from the light source unit 42 and guided by the light guide unit 26, have the same relative intensity with respect to a light distribution angle at a position where the illumination light has passed through the lens member 53, specifically, at the emission end 28 of the second light guide 52. That is, a variation in relative intensity can be suppressed. The same relative intensity mentioned here means that a difference in the relative intensities of the respective color lights of the illumination light with respect to a light distribution angle is very small, and it is preferable that all the relative intensities of a violet light V, a blue light B, and a red light R have a difference of ±5% or less from the relative intensity of a green light G in a case where the relative intensity of a green light G is used as a reference.

As described above, in this embodiment, the light guide unit 26 includes the first light guide 51, the second light guide 52, and the lens member 53, and the first light guide 51, the second light guide 52, and the lens member 53 are fixed in the first light guide rod 74 having stiffness. Accordingly, the positions of optical axes of the first light guide 51, the second light guide 52, and the lens member 53 are not shifted from each other. For this reason, the light guide unit 26 can prevent a reduction in the amount of illumination light and deterioration in the distribution of the illumination light via the lens member 53 regardless of the operation state of the endoscope 12.

Further, the endoscope 12 needs to be subjected to sterilization treatment whenever being used. In this case, it is common to perform sterilization treatment using an autoclave. During this sterilization treatment, the connector 25 including the light guide rod 65 is exposed to saturated water vapor having high temperature and high pressure (for example, 135° C. and 5 atmospheres) for about 20 minutes.

In a case where the lens member 53, the first light guide 51, and the second light guide 52 are in contact with each other and sterilization treatment using an autoclave is repeatedly performed, there is a possibility that the lens member 53 may be damaged by being pressed from the first and second light guides 51 and 52 due to a difference between the thermal expansion coefficient of the lens member 53 and the thermal expansion coefficients of the first and second light guides 51 and 52. However, since the lens member 53 has the gaps D1 and D2 from end surfaces of the first and second light guides 51 and 52 in the present invention, the lens member 53 and the first and second light guides 51 and 52 are not in contact with each other even though the endoscope 12 is subjected to sterilization treatment. For this reason, the lens member 53 is not damaged. That is, even though sterilization treatment is repeatedly performed, a reduction in the amount of illumination light and deterioration in the distribution of the illumination light caused by the light guide unit 26 can be prevented. Further, since the lens member 53 has the gaps D1 and D2, an adhesive used to fix the first and second light guides 51 and 52 does not adhere to the lens member 53.

### Second Embodiment

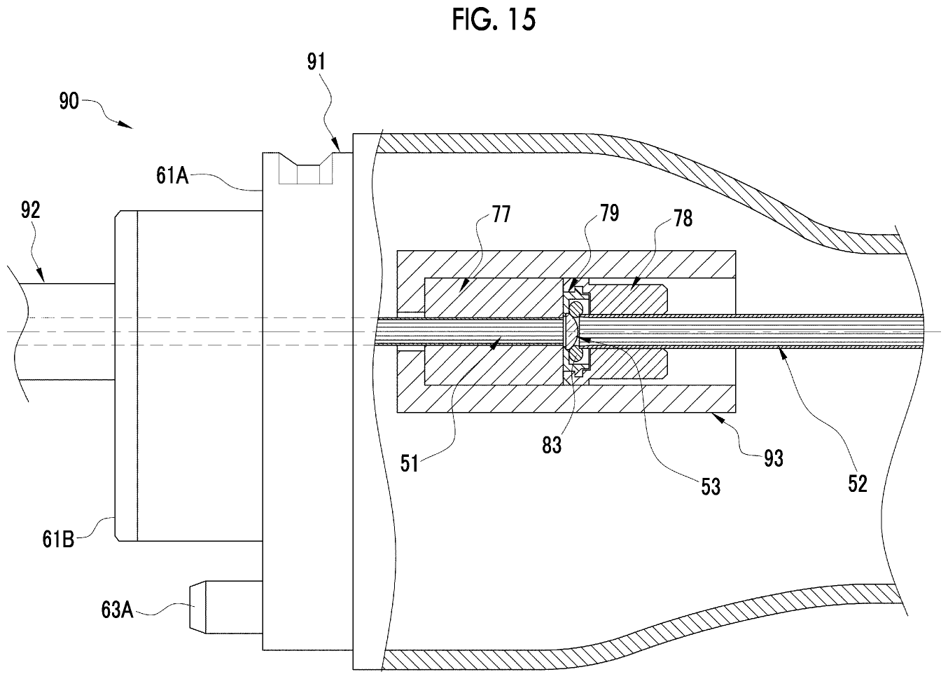

An example in which the lens member 53 for preventing a reduction in the amount of light and deterioration in the distribution of light is disposed in the light guide rod 65 has been described in the first embodiment. However, the present invention is not limited thereto, and the lens member 53 may be disposed in a portion of the connector 25 other than the light guide rod 65. In an example shown in FIG. 15, the lens member 53 is disposed in an outer case 91 of a connector 90. A configuration except for the connector 90 is the same as the configuration of the endoscope system 10 according to the first embodiment, and the same components and the like as in the first embodiment will be denoted by the same reference numerals as those in the first embodiment, and the detailed description thereof will be omitted.

As with the connector 25 of the first embodiment, the connector 90 is provided at a proximal end portion of the universal cable 23 and is attachably and detachably connected to the light source device-side connector 41 of the light source device 14. The connector 90 comprises the outer case 91, a light guide rod 92, and a lens member holding portion 93. As with the outer case 61 of the connector 25 of the first embodiment, the outer case 91 houses the wireless communication unit 63, the wireless power receiving unit 64, and the like, and a light guide rod 92 protrudes from a front surface 61A that is positioned on a side facing the light source device 14. Unlike the light guide rod 65 of the connector 25 of the first embodiment, the light guide rod 92 holds only the first light guide 51 and does not hold the second light guide 52 and the lens member 53.

As in the first light guide rod 74 of the first embodiment, the first light guide 51, the second light guide 52, and the lens member 53 are fixed in the lens member holding portion 93 via the first ferrule 77, the second ferrule 78, and the spacer 79. Accordingly, the lens member 53 is disposed between the first and second light guides 51 and 52. Further, as in the first embodiment, the incident end 51A of the first light guide 51 faces the light source unit 42, and the emission end 28 of the second light guide 52 is disposed at the distal end portion 21A. Accordingly, the light guide unit 26 guides illumination light, which is generated from the light source unit 42, up to the distal end portion 21A of the endoscope 12. Furthermore, the O-ring 83 is housed in the spacer 79, and the O-ring 83 is interposed between the lens member 53 and the second light guide 52 as in the first embodiment. For this reason, the lens member 53 and the second light guide 52 are disposed to have an interval therebetween.

The lens member holding portion 93 is fixed in the outer case 91. The outer case 91 and the lens member holding portion 93 are formed of, for example, resin components and have high stiffness. Further, an O-ring or the like (not shown) is provided in a gap between the components of the outer case 91, so that airtightness is ensured. Accordingly, the lens member holding portion 93 does not require a configuration in which airtightness is ensured. That is, since the O-rings 81, 82, and 84 and the like used to ensure airtightness do not need to be provided unlike in the first light guide rod 74 of the first embodiment, the number of components can be reduced.

Since the outer case 91 and the lens member holding portion 93 have stiffness, the positions of the optical axes of the first light guide 51, the second light guide 52, and the lens member 53 are not shifted from each other. For this reason, as in the first embodiment, the light guide unit 26 can prevent a reduction in the amount of illumination light and deterioration in the distribution of the illumination light via the lens member 53 regardless of the operation state of the endoscope.

### Third Embodiment

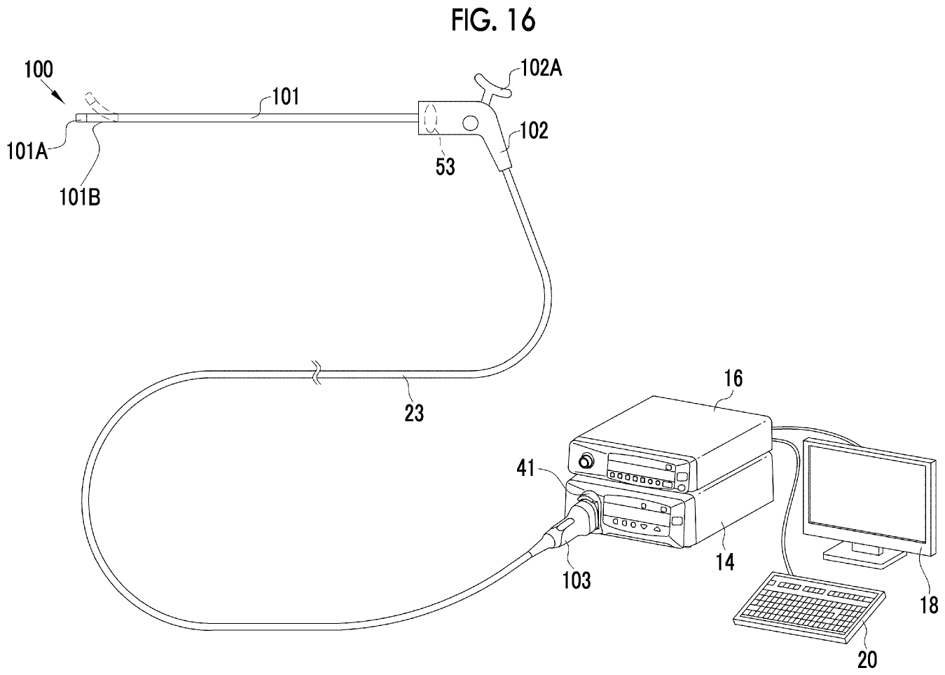

Examples in which the lens member 53 for preventing a reduction in the amount of light and deterioration in the distribution of light is disposed in the connectors 25 and 90 have been described in the first and second embodiments. However, the present invention is not limited thereto, and the lens member 53 may be disposed in other components of the endoscope having stiffness. In an example of an endoscope 100 shown in FIG. 16, an operation part 102 connected to an insertion part 101 is provided, and the lens member 53 is disposed in the operation part 102. A configuration except for the endoscope 100 is the same as the configuration of the endoscope system 10 according to the first embodiment, and the same components and the like as in the first embodiment will be denoted by the same reference numerals as those in the first embodiment, and the detailed description thereof will be omitted.

As with the endoscope 12 of the first embodiment, the endoscope 100 is, for example, a rigid endoscope, such as a laparoscope, and comprises an elongated rigid insertion part 101 that is to be inserted into an object to be examined, an operation part 102 that is connected to a proximal end portion of the insertion part 101, and a soft universal cable 23 that is connected to the operation part 102. A connector 103 is provided at a proximal end portion of the universal cable 23, and the endoscope 100 is attachably and detachably connected to the light source device-side connector 41 of the light source device 14 via the connector 103. Unlike the connectors 25 and 90 of the first and second embodiments, the connector 103 holds only the first light guide 51 and does not hold the second light guide 52 and the lens member 53.

The insertion part 101 includes a distal end part 101A and a bendable part 101B. As with the distal end portion 21A of the endoscope 12 of the first embodiment, the distal end part 101A is provided with the observation unit 27 and the emission end 28. The bendable part 101B is connected to the distal end part 101A and is provided to be bendable. The operation part 102 is provided with a bending operation lever 102A, operation buttons (not shown), and the like. The bending operation lever 102A is an operation member that is used to bend the bendable part 101B. Since the bendable part 101B is bent, the orientation of the distal end part 101A can be changed.

The operation part 102 is formed of, for example, a resin component and has high stiffness. It is preferable that the lens member 53 is provided in the endoscope 100 at a portion other than the bendable part 101B. More specifically, the lens member 53 is provided in the operation part 102. As with the lens member holding portion 93 of the second embodiment, a holding portion (not shown) in which the first light guide 51, the second light guide 52, and the lens member 53 are fixed is integrally provided in the operation part 102.

In a case where the lens member 53 is provided in the bendable part 101B and the bendable part 101B is bent, the positions of the optical axes of the first light guide 51, the second light guide 52, and the lens member 53 are shifted from each other. On the other hand, the lens member 53 is provided in the operation part 102 in this embodiment. Since the operation part 102 has stiffness, the positions of the optical axes of the first light guide 51, the second light guide 52, and the lens member 53 are not shifted from each other. For this reason, as in the first embodiment, the light guide unit 26 can prevent a reduction in the amount of illumination light and deterioration in the distribution of the illumination light via the lens member 53 regardless of the operation state of the endoscope.

An endoscope to be used as a laparoscope has been described in the above-mentioned embodiments by way of example, but the present invention can also be applied to, for example, endoscopes used for other uses, such as an industrial use, and the like.

### EXPLANATION OF REFERENCES

10: endoscope system
12: endoscope
14: light source device
16: processor device
18: monitor
20: console
21: insertion part
21A: distal end portion
22: grip part
23: universal cable
24: switch-disposition member
24A: operation switch
25: connector
26: light guide unit
27: observation unit
28: illumination light-emission end
29: observation window
31: image pickup lens group
32: prism
33: image pickup sensor
41: light source device-side connector
41A: locking portion
41B: locking portion
41C: contact surface
41D: fitting recess
41E: connection hole
42: light source unit
42a: V-LED
42b: B-LED
42c: G-LED
42d: R-LED
43: light source controller
44: wireless communication unit
44A: connection hole
45: wireless power supply unit
46: image signal receiving unit
51: first light guide
51A: incident end
51B: emission end
52: second light guide
52A: incident end
53: lens member
53A, 53B: lens surface
61: outer case
61A: front surface
61B: fitting projection
61C: upper surface
61D: lower surface
61E, 61F: groove
61G: lead-out hole
62: shield case
63: wireless communication unit
63A: connection pin
64: wireless power receiving unit
65: light guide rod
66: image signal transmission unit
68: case body
69: case lid
71: O-ring
72: bracket
73: O-ring
74: first light guide rod
75: second light guide rod
75A: fitting portion
75B: end portion
76: incident window
77: first ferrule
77A: light guide-insertion hole
78: second ferrule
78A: light guide-insertion hole
78B: opening portion
78C: locking groove
78D: large-diameter portion
78E: small-diameter portion
79: spacer
79A: through-hole
79B: lens member holding portion
79C: O-ring housing portion
79D: outer peripheral surface
79E: locked portion
81, 82, 83, 84: O-ring
90: connector
91: outer case
92: light guide rod
93: lens member holding portion
100: endoscope
101: insertion part
101A: distal end part
101B: bendable part
102: operation part
102A: bending operation lever
103: connector
D1, D2: gap
L_{LG}: central axis
P: focal point

What is claimed is:

1. An endoscope system comprising:
an endoscope that includes an insertion part to be inserted into an object to be examined, an illumination light-emission end provided at a distal end portion of the insertion part, and a light guide unit which guides illumination light; and
a light source device that is connected to the endoscope, includes a plurality of light sources emitting color lights different from each other, and emits illumination light in which a plurality of color lights are mixed by the light sources,
wherein the light guide unit includes a light guide that guides the illumination light emitted from the light sources to the illumination light-emission end in a case where the endoscope is connected to the light source device, and an optical member that suppresses a variation in a relative intensity of each color light of the illumination light, which is guided by the light guide and is emitted from the illumination light-emission end, with respect to a light distribution angle, and
the optical member is provided in the endoscope at a portion that is closer to a proximal end side than to the illumination light-emission end and that has stiffness.

2. The endoscope system according to claim 1,
wherein the light guide unit includes a plurality of the light guides and the optical member is a lens member disposed between the plurality of light guides.

3. The endoscope system according to claim 2,
wherein the light guide and the lens member are disposed at a position where an emission point of the illumination light guided by the light guide is closer to the lens member than to a focal point of the lens member on an incident side.

4. The endoscope system according to claim 2,
wherein lens surfaces of the lens member are disposed to have intervals from an emission end and an incident end of the light guides.

5. The endoscope system according to claim 1,
wherein the endoscope includes a connector to be connected to the light source device, and
the optical member is disposed in the connector.

6. The endoscope system according to claim 1,
wherein the endoscope includes a connector to be connected to the light source device,
the connector includes a light guide rod that is to be inserted into the light source device in a case where the connector is connected to the light source device, and
the optical member is disposed in the light guide rod.

7. The endoscope system according to claim 1,
wherein the insertion part includes a bendable part that changes an orientation of the distal end portion, and
the optical member is provided in the endoscope at a portion other than the bendable part.

8. The endoscope system according to claim 1,
wherein the endoscope includes an operation part connected to the insertion part, and
the optical member is provided in the operation part.

9. The endoscope system according to claim 2,
wherein a dimension from an incident end to an emission end of a light guide, which is positioned on an incident side of the optical member, among the plurality of light guides in a direction of an optical axis is 5 mm or more.

10. The endoscope system according to claim 2,
wherein an antireflection film is formed on a lens surface of the lens member.

11. The endoscope system according to claim 2,
wherein in a case where a relative intensity of one color light among the plurality of color lights emitted from the light sources is used as a reference, the lens member makes a relative intensity of the other color light have a difference of ±5% or less from the relative intensity of the color light used as the reference.
